(19) United States
(12) Patent Application Publication
(10) Pub. No.: US 2009/0136554 A1
(43) Pub. Date: May 28, 2009

(54) Transdermal sustained release drug delivery

(75) Inventors: Mahmoud Ameri, Fremont, CA (US); Yuh-Fun Maa, Millbrae, CA (US)

Correspondence Address: Edwards Angell Palmer & Dodge LLP, P.O. Box 55874, Boston, MA, 02205, US

(73) Assignee: Alza Corporation, Mountain View, CA (US)

(21) Appl. No.: 12/288,753

(22) Filed: Oct. 23, 2008

Related U.S. Application Data
(60) Provisional application No. 60/982,119, filed on Oct. 23, 2007.

(51) Int. Cl. A61F 2/00

(52) U.S. Cl. 424/422

(57) ABSTRACT

Provided herein are microprojections and microprojection arrays for delivering biologically active agents. Also provided herein are compositions suitable for coating such microprojections and microprojection arrays.

### FIELD OF THE PRESENT INVENTION

The present invention relates generally to biologically active agent compositions, devices for delivering biologically active agents and methods for formulating such compositions and delivering such agents. More particularly, the present invention relates to transdermal sustained release drug delivery compositions and devices.

### BACKGROUND OF TH INVENTION

A great number and variety of biologically active agents are known in the art to have therapeutic benefits when delivered appropriately to a patient having a condition upon which such biologically active agents can exert a beneficial effect. These biologically active agents comprise several broad classes, including, but not limited to peptides or proteins, such as hormones, proteins, antigens, repressors/activators, and enzymes, among others. Therapeutic applications include treatment of diabetes, cancer, hypercalcemia, Paget's disease, osteoporosis, diabetes, cardiac conditions, including congestive heart failure, sleep disorders, Chronic Obstructive Pulmonary Disease (COPD) and anabolic conditions, to name a few.

Biologically active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent intravenously or subcutaneously, while assuring no modification of the agent during administration, is a difficult, inconvenient, painful and uncomfortable procedure that sometimes results in poor patient compliance.

Hence, in principle, transdermal delivery provides for a method of administering active agents that would otherwise need to be delivered via hypodermic injection or intravenous infusion. The word “transdermal”, as used herein, is generic term that refers to delivery of a biologically active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal protein flux has been limited, at least for the larger proteins, due to their size.

There also have been many techniques and devices developed to mechanically penetrate or disrupt the outermost skin layers thereby creating pathways into the skin in order to enhance the amount of agent being transdermally delivered. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is possible to have the active agent that is to be delivered coated on the microprojections instead of contained in a physical reservoir. This eliminates the necessity of a separate physical reservoir and developing an agent formulation or composition specifically for the reservoir.

### SUMMARY OF THE INVENTION

In certain embodiments, the present invention provides a transdermal delivery device for delivering a biologically active agent comprising at least one stratum corneum-piercing microprojection, wherein said microprojection has a first coating comprising said biologically active agent and a second coating comprising a polymer, wherein the polymer coating allows controlled release of said biological agent after the transdermal delivery device is applied to and/or into the skin of a subject. The active agent is preferably glucagon-like peptides (GLP) and analogs thereof, such as GLP-1, GLP-2, and analogs thereof. The active agent can include exendin-4 or an exenatide based agent.

In some embodiments, the present invention provides for a transdermal delivery device for delivering a biologically active agent comprising at least one stratum corneum-piercing microprojection, wherein said microprojection has a plurality of coating layers; wherein at least one coating layer comprises said biologically active agent (e.g., an exendin-4 or exenatide based agent) and at least one coating layer comprises a polymer, wherein the polymer coating allows controlled release of said biological agent after the transdermal delivery device is applied to and/or into the skin of a subject. In some embodiments, the coating layers comprising the biologically active agent and coating layers comprising the controlled release polymer are alternately disposed on said microprojection. The active agent is preferably glucagon-like peptides (GLP) and analogs thereof, such as GLP-1, GLP-2, and analogs thereof. The active agent can include exendin-4 or an exenatide based agent. In some embodiments, the coating is an amorphous glass coating. Father in other embodiments, the coating is a co-formulation of the active agent with the polymer. This co-formulation can be used by itself or in combination with the sequential layers.

In certain embodiments, the polymer or controlled release polymer used in the polymer coating is a hydrophilic polymer or a hydrophobic PLGA copolymer. In various embodiments of the present invention, the polymer layer has a thickness selected to provide a predetermined sustained release profile for the biologically active agent. In some embodiments, the polymer layer has a copolymer molar mass, a copolymer architecture, a water hydration rate, and/or or layer thickness selected to provide a predetermined sustained release profile for the biologically active agent.

In certain embodiments, the device provided herein has a controlled release profile with a shorter tₘₐₓ and a rapid concentration drop off. In some embodiments of the present invention, the controlled release profile of a device has a reduced Cₘₐₓ and an extended drop-off tail. It is to be understood that these pharmacokinetic values are compared to those of standard applications of the biologically active agent.

In certain embodiments, the polymer layer encapsulates the biologically active agent and slows down release of the biologically active agent.

In some embodiments, the biologically active agent is selected from an exendin-4 or exenatide based agent. In certain embodiments, the exenatide based agent is exenatide. In some embodiments, the exenatide based agent is an exenatide salt. In specific embodiments, the exenatide salt comprises a non-volatile counter-ion.

in some embodiments, the first coating (or coating comprising the biologically active agent) comprises a first and a second exenatide based agent. In some embodiments, the second exenatide based agent is a second exenatide salt, wherein the second exenatide salt comprises a volatile counter-ion. In specific embodiments, the first exenatide based agent is a first exenatide salt and the second exenatide based agent is a second exenatide salt. In more specific embodiments, the first exenatide salt is a non-volatile salt and the second exenatide salt is a volatile salt.

In other embodiments, the first exenatide based agent is an exenatide salt and the second exenatide based agent is a net neutral species of exenatide. In certain embodiments, the net neutral species of exenatide is obtained from an exenatide salt comprising a volatile counter-ion upon volatilization of the volatile counter-ion. In other embodiments, the net neutral species of exenatide is simply combined with the first exenatide based agent (e.g., an exenatide salt).

In certain embodiments, the present invention provides for a transdermal delivery device comprising at least one stratum corneum-piercing microprojection. In some embodiments, the microprojection has a coating layer; the coating layer has a first exenatide based agent and a second exenatide based agent. In some embodiments, the second exenatide based agent is released in a controlled manner. In certain embodiments, the first exenatide based agent is an exenatide salt with a non-volatile counter-ion and the second exenatide based agent is a net neutral species of exenatide.

In various embodiments of the present invention, the microprojection or microprojections of the devices provided herein have a length of less than about 500 micrometers and a thickness of less than about 25 micrometers. In certain embodiments, stratum corneum-piercing microprojection or microprojections are formed by etching the microprotrusion from a thin sheet and folding said microprojection out of a plane of the sheet.

Certain embodiments of the present invention provide for a composition for coating a transdermal delivery device having stratum corneum-piercing microprojections. In some embodiments, the composition comprises a formulation of a biologically active agent (e.g., an exendin-4 or exenatide based agent), a non-volatile counter-ion and a volatile counter-ion. In some embodiments, the non-volatile counter-ion causes the formulation of a first species of exendin-4 or exenatide based agent that has improved solubility when the formulation is dried. In certain embodiments, the volatile counter-ion causes the formation of a second species of exendin-4 or exenatide based agent that has reduced solubility when the formulation is dried. In some embodiments, the first species is adapted to rapidly provide a therapeutically relevant blood level of said biologically active agent when said formulation is allowed to dissolve in a bodily fluid. In certain embodiments, the second species is adapted to provide a sustained therapeutically relevant blood level of said biologically active agent when said formulation is allowed to dissolve in a bodily fluid.

In some embodiments of the present invention, the composition contains approximately equimolar amounts of said non-volatile counter-ion and said volatile counter-ion. In specific embodiments, the formulation has a pH, the exenatide based agent has a positive charge at said formulation pH and the non-volatile counter-ion comprises a non-volatile weak acid. In certain embodiments, non-volatile weak acids useful herein have an acidic pKa and a property selected from the group consisting of a melting point higher than about 50° C. and a boiling point higher than about 170° C. at atmospheric pressure. In specific embodiments, the non-volatile weak acid is selected from, by way of non-limiting example, citric acid, succinic acid, glycolic acid, gluconic acid, glucuronic acid, lactic acid, malic acid, pyruvic acid, tartaric acid, tartronic acid, and fumaric acid.

In other embodiments, the formulation has a pH, the exenatide based agent has a positive charge at the formulation pH and the non-volatile counter-ion comprises a strong acid. In certain embodiments, the strong acid has at least one pKa lower than about 2. In specific embodiments, the strong acid is selected from, by way of non-limiting example, hydrochloric acid, hydrobromic acid, nitric acid, sulfonic acid, sulfuric acid, maleic acid, phosphoric acid, benzene sulfonic acid and methane sulfonic acid.

In still other embodiments, the formulation has a pH, the exenatide based agent has a positive charge at the formulation pH and said non-volatile counter-ion comprises an acidic zwitterion. In certain embodiments, the acidic zwitterion has at least two acidic pKas and at least one basic pKa, so that there is at least one acidic pKa more than said basic pKas. In specific embodiments, the acidic zwitterion is selected from, by way of non-limiting example, glutamic acid and aspartic acid.

In yet other embodiments, the formulation has a pH, the exenatide based agent has a negative charge at said formulation pH and the non-volatile counter-ion comprises a non-volatile weak base. In certain embodiments, the non-volatile weak base has a basic pKa and a property selected from the group consisting of a melting point higher than about 50° C. and a boiling point higher than about 170° C. at atmospheric pressure. In specific embodiments, the non-volatile weak base is selected from, by way of non-limiting example, monoethanolomine, diethanolamine, triethanolamine, tromethamine, methylglucamine, glucosamine.

In still other embodiments, the formulation has a pH, the exenatide based agent has a negative charge at said formulation pH and said non-volatile counter-ion comprises a strong base. In certain embodiments, the strong base has at least one pKa higher than about 12. In specific embodiments, the strong base is selected from, by way of non-limiting example, of sodium hydroxide, potassium hydroxide, calcium hydroxide, and magnesium hydroxide.

In other embodiments, the formulation has a pH, the exenatide based agent has a negative charge at the formulation pH and said non-volatile counter-ion comprises a basic zwitterion. In certain embodiments, the basic zwitterion has at least two basic pKas and at least one acidic pKa, so that there is at least one basic pKa more than acidic pKas. In specific embodiments, the basic zwitterion is selected from, by way of non-limiting example, histidine, lysine, and arginine.

In some embodiments, the formulation has a pH, the exenatide based agent has a positive charge at said formulation pH and said non-volatile counter-ion comprises a mixture of counter-ions. In certain embodiments, one counter-ion is a non-volatile strong acid and another counter-ion is a non-volatile weak acid. In certain embodiments, the formulation has a pH, the exenatide based agent has a negative charge at said formulation pH and said non-volatile counter-ion comprises a mixture of counter-ions comprising at least one non-volatile strong base and at least one non-volatile weak base.

In additional embodiments, the volatile counter-ion comprises a mixture of counter-ions.

In some embodiments, the formulation has a pH, the exenatide based agent has a positive charge at said formulation pH and said volatile counter-ion comprises a volatile weak acid. In certain embodiments, the volatile weak acid has an acidic pKa higher than about 2 and a property selected from the group consisting of a melting point lower than about 50° C. and a boiling point lower than about 170° C. at Pₐₜₘ. In specific embodiments, the volatile weak acid is selected from, by way of non-limiting example, acetic acid, propionic acid and pentanoic acid.

In other embodiments, the formulation has a pH, the exenatide based agent has a negative charge at the formulation pH and the volatile counter-ion comprises a volatile weak base. In certain embodiments, the volatile weak acid has a basic pKa lower than approximately 12 and a property selected from the group consisting of a melting point lower than about 50° C. and a boiling point lower than about 170° C. at Pₐₜₘ. In specific embodiments, the volatile weak base is selected from, by way of non-limiting example, ammonia and morpholine.

In certain embodiments of the present invention, the formulation includes said exenatide-based agent in the range of about 1-60 wt. % of said formulation. In more specific embodiments, the formulation includes said exendin-4 or exenatide-based agent in the range of about 5-30 wt. % of said formulation. In some embodiments, the formulation has a pH in the range of about 1-6. In more specific embodiments, the formulation has a pH in the range of about 2-5.5. In some embodiments, the formulation further contains a formulation adjuvant. In specific embodiments, the adjuvant is selected from, by way of non-limiting example, a buffer, an antioxidant, a surfactant, an amphiphilic polymer, a hydrophilic polymer, a biocompatible carrier, a stabilizing agent, a vasoconstrictor, a pathway patency modulator, a solubilising/complexing agent, a non-aqueous solvent, an aqueous solvent, or combinations thereof.

In some embodiments, the formulation has a viscosity of about 3 to about 500 centipoise.

In certain embodiments, the present invention provides for a transdermal delivery device comprising at least one stratum corneum-piercing microprojection, wherein said microprojection is coated with a composition as described herein. In some embodiments, the composition is dried.

In certain embodiments of the present invention, the microprojection are coated with a biocompatible coating (including coatings formed from the formulations and/or compositions provided herein) that has a thickness of less than about 25 microns. In more specific embodiments, the biocompatible coating has a thickness less than approximately 10 microns. In some embodiments, on top of the biocompatible coating (including coatings formed from the formulations and/or compositions provided herein) is an additional coating layer. In some embodiments, the additional coating layer comprises a polymer. In certain embodiments, the additional polymer containing coating layer allows for controlled release of said biological agent after the transdermal delivery device is applied to the skin of a subject.

In other embodiments, the present invention provides for a method for transdermally delivering a biologically active agent (e.g., an exendin-4 or exenatide based agent) comprising the steps of: providing a transdermal delivery device having at least one stratum corneum-piercing microprojection, the microprojection including a biocompatible coating comprising a dried formulation of said exenatide based agent, a non-volatile counter-ion and a volatile counter-ion. In certain embodiments, the non-volatile counter-ion causes the formation of a first species of exendin-4 or exenatide based agent that has improved solubility when said formulation is dried and the volatile counter-ion causes the formation of a second species of exendin-4 or exenatide based agent that has reduced solubility when said formulation is dried; and applying said delivery device to a patient to deliver said biologically active agent.

In certain embodiments of the present invention, methods provided herein contain the step of rapidly establishing a therapeutically relevant blood level of said agent in said patient by dissolving the first species of biologically active agent (e.g., an exendin-4 or exenatide based agent). In some embodiments, the step of rapidly establishing a therapeutically relevant blood level of the agent comprises establishing the relevant blood level in less than 60 min after applying the device. In some embodiments, the step of rapidly establishing a therapeutically relevant blood level of the agent comprises establishing the relevant blood level in less than 30 min after applying the device. In more specific embodiments, the step of rapidly establishing a therapeutically relevant blood level of the agent comprises establishing said blood level in less than 15 min after applying the device. In some embodiments, the present invention provides for the step of maintaining a therapeutically relevant blood level of said agent in said patient by dissolving said second species of biologically active agent (e.g., an exendin-4 or exenatide based agent). In some embodiments, the step of maintaining a therapeutically relevant blood level of the agent comprises maintaining said blood level in the range of about 1 to 6 hours. In more specific embodiments, the step of maintaining a therapeutically relevant blood level of the agent comprises maintaining said blood level in the range of about 2 to 4 hours. In certain embodiments, the therapeutically relevant blood level is plasma level of greater than about 50 pg/mL. In the preferred embodiments, the blood level is maintained for about 24 hours.

In certain embodiments of the present invention, the amount of exendin-4 or exenatide based agent delivered in the range of approximately 1 to 1000 μg per day.

In some embodiments, the present invention provides for a method for applying a biocompatible coating to a transdermal delivery device that has a least one stratum corneum-piercing microprojection. In some embodiments, the method of applying the biocompatible coating comprises the steps of: providing a formulation of a biologically active agent (e.g., an exendin-4 or exenatide based agent), a non-volatile counter-ion, and a volatile counter-ion; applying the formulation to said microprojection; and drying the formulation. In certain embodiments, the non-volatile counter-ion causes the formation of a first species of exendin-4 or exenatide based agent that has improved solubility when the formulation is dried and the volatile counter-ion causes the formation of a second species of exendin-4 or exenatide based agent that has reduced solubility when said formulation is dried.

In other embodiments, the present invention provides a method of preparing a transdermal delivery device comprising: providing an biocompatible formulation of a biologically active agent (e.g., an exendin-4 or exenatide based agent); applying the biocompatible formulation to said microprojection; drying the biocompatible formulation to form a microprojection coated with a biocompatible coating; providing a controlled release formulation comprising a polymer (e.g., a polymer suitable for providing controlled and/or sustained release); applying the controlled release formulation to the microprojection coated with a biocompatible coating; drying the controlled release formulation. In some embodiments, the biocompatible formulation contains a biologically active agent (e.g., an exendin-4 or exenatide based agent), a non-volatile counter-ion, and a volatile counter-ion.

In specific embodiments of the present invention, the biologically active agent is an exendin-4 or exenatide based agent. In more specific embodiments of the present invention, the exendin-4 or exenatide based agent is a pharmaceutically acceptable salt. In some embodiments of the present invention, the exenatide based agent is exenatide (or a net neutral species of exenatide). In some embodiments of the present invention, the exenatide based agent is an exenatide salt comprising a volatile counter-ion or mixtures of volatile counter-ions. In some embodiments of the present invention, the exenatide based agent is an exenatide salt with a non-volatile counter-ion or mixtures of non-volatile counter-ions. In some embodiments of the exenatide based agent is a mixture of exenatide salts having one or more volatile counter-ions and one or more non-volatile counter-ions.

In certain embodiments, the present invention provides for a method for forming a device for transdermally delivering a biologically active agent (e.g., an exenatide based agent) comprising the steps of: forming at least one stratum corneum-piercing microprojection in a thin sheet of material; applying a first coating comprising a biologically active agent and a second coating comprising a polymer, wherein the polymer coating allows controlled release of said biological agent after the transdermal delivery device is applied to the skin of a subject. In certain embodiments, the method further includes the step of bending said microprojection out of a plane formed by said thin sheet after applying said first and second coatings.

In certain embodiments, the microprojections described herein are formed by, for example, etching and punching.

In some embodiments, the present invention provides for a transdermal delivery device for delivering a biologically active agent comprising a microprojection array of a plurality of stratum corneum-piercing microprojections, wherein at least a portion of each of said microprojections has a first coating comprising said biologically active agent and a second coating comprising a polymer, wherein the polymer coating allows controlled release of said biological agent after the transdermal delivery device is applied to the skin of a subject.

Certain embodiments of the present invention provide for a device comprising a plurality of microprojections. In some embodiments, the microprojections are arranged in a microprojection array. In some embodiments, the array has a density of at least about 10 microprojections/cm². In specific embodiments of the present invention, the microprojection array has a density of about 200-2000 microprojections/cm².

In some embodiments, the present invention provides for a transdermal delivery device for delivering a biologically active agent. The devices has a microprojection array containing a plurality of stratum corneum-piercing microprojections. At least a portion of each of said microprojections has a coating containing the biologically active agent, a hydrophilic counter-ion and a hydro-phobic counter-ion. In some embodiments, the biologically active agent is exenatide. In various embodiments, the hydrophilic counter-ion is acetate. In certain embodiments, the hydrophobic counter-ion is selected from hexadecanoate, pentadecanoate, tetradecanoate, tridecanoate, dodecanoate, decanoate, nonanoate, octanoate, hetpanoate, hexanoate, pentanoate and butanoate. In some embodiments, the hydrophobic counter-ion is selected from protonated ethylamine, propylamine and butylamine.

In certain embodiments, the present invention provides for a transdermal delivery device that delivers a biologically active agent in an amount sufficient to provide a therapeutically relevant dose of the biologically active agent within about 30 to about 60 minutes.

In some embodiments, the transdermal delivery device is suitable for delivering a therapeutically effective amount of biologically active agent. In certain embodiments, the biologically active agent is an exenatide based agent. In various embodiments, the exenatide based agent is exenatide and/or pharmaceutically acceptable salt or salts thereof. In some embodiments, the therapeutically effective amount of exenatide and/or pharmaceutically acceptable salt or salts thereof is about 24 μg. In certain embodiments, the therapeutically effective amount of exenatide and/or pharmaceutically acceptable salt or salts thereof is delivered over a period of about 6 hours. In some embodiments, the exenatide and/or pharmaceutically acceptable salt or salts thereof is delivered in an amount sufficient to provide an AUC of the exenatide and/or pharmaceutically acceptable salt or salts thereof between about 600 pg·h/mL and about 950 pg·h/mL. In some embodiments, the exenatide and/or pharmaceutically acceptable salt or salts thereof is delivered in an amount sufficient to provide a Cₘₐₓ of the exenatide and/or pharmaceutically acceptable salt or salts thereof between about 210 pg/mL and about 220 pg/mL.

Preferably, in some embodiments, the delivery of exenatide is prolonged to achieve a once a day drug exposure similar to once weekly. In some embodiments, the once a day patch delivers the equivalent of 1/7^{th} exposure of Byetta LAR and at a minimum matches the exposure with the twice daily injection of Byetta. Such delivery profiles which reduce Cmax with the polymer encapsulation preferably inhibit the nausea and other side effects associated with Byetta. Also, prolonged exposure of exenatide can improve weight loss benefit, provide better control of glucose and be better at promoting insulin production from the pancreas. Thus, preferably, improved pharmacokinetic profiles provides for a more desirable pharmacodynamic effect.

In a preferred embodiment, the following product profile is present in a product comprising exenatide—1) dosing regimen of single patch application per day; 2) efficacy and safety profile comparable to marketed Byetta product; 3) PK profile—AUC of about 600—about 950 pg*h/ml and steady state of about 210—about 220 pg/ml; and/or 4) with a delivery to match 10 mcg BID regimen, ˜24 mcg of exenatide is delivered over a period of ˜6 hours. A preferred plasma level is depicted in FIG. 12B.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member, according to the invention;

FIG. 2 is a perspective view of the microprojection member shown in FIG. 1 having a coating deposited on the microprojections, according to the invention;

FIG. 3 is a side sectional view of a microprojection member having an adhesive backing, according to the invention;

FIG. 4 is a side sectional view of a retainer having a microprojection member disposed therein, according to the invention;

FIGS. 5 and 6 are a perspectives view of the retainer shown in FIG. 4;

FIG. 7 shows a microprojection with alternating layers of drug formulation coating and sustained release polymer coating;

FIG. 8 shows a microprojection array with an exenatide drug formulation coating only prior to and after being exposed to PBS for 1 minute;

FIG. 9 shows a microprojection array of an exenatide formulation coating encapsulated with a polymer coating prior to and after being exposed to PBS for 1 minute;

FIG. 10 shows a microprojection array of an exenatide formulation coating encapsulated with a polymer coating prior to and after being exposed to PBS for 5 minutes;

FIG. 11 shows a microprojection array of an exenatide formulation coating encapsulated with a polymer coating prior to and after being exposed to PBS for 10 minutes; and

FIG. 12A shows the charge profile of Exendin-4.

FIG. 12B shows a dosing model for plasma levels of exenatide.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### DEFINITIONS

The term “transdermal”, as used herein, means the delivery of a biologically active agent into and/or through the skin for local or systemic therapy.

The term “transdermal flux”, as used herein, means the rate of transdermal delivery.

The terms “pulsatile delivery profile” and “pulsatile concentration profile”, as used herein, mean a post administration increase in blood serum concentration of a biologically active agent from a baseline concentration to a concentration in the range of approximately 10-1000 pg/mL in a period ranging from 1 min. to 4 hr., wherein Cₘₐₓ is achieved, and a decrease in blood serum concentration from Cₘₐₓ to the baseline concentration in a period ranging from 1-8 hrs. after Cₘₐₓ has been achieved.

Other concentration profiles resulting in a pulsatile delivery comprising a rise in blood concentration of the biologically active agent to a Cₘₐₓ of 50-1000 pg/mL within a twelve-hour period following administration would also likely result in the desired beneficial effect and, hence, are within the scope of the present invention.

The term “co-delivering”, as used herein, means that a supplemental agent(s) is administered transdermally either before the biologically active agent is delivered, before and during transdermal flux of the biologically active agent during transdermal flux of the biologically active agent during and after transdermal flux of the biologically active agent and/or after transdermal flux of the biologically active agent. Additionally, two or more biologically active agents of a similar type (e.g., two or more exenatide based agents) may be formulated in the coatings and/or formulations, resulting in co-delivery of the biologically active agents.

The term “exenatide based agent”, as used herein, includes, without limitation, exenatide salts, exenatide analogs and closely related peptides and agents including but not limited to glucagons like peptide 1 (GLP-1) and analogs having a peptide sequence that functions by the same means as the biologically active region of exenatide.

Examples of suitable exenatide salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, \tilde{□}hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

The noted exenatide based agents can also be in various forms, such as net neutral species (e.g., non-charged or zwitterionic species), free bases, acids, charged or uncharged molecules, components of molecular complexes or nonirritating, pharmacologically acceptable salts. Exenatide (exendin-4) has the structure: H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH.

It is to be understood that more than one biologically active agent (e.g., exendin-4 or exenatide based agent) can be incorporated into the agent source, reservoirs, and/or coatings of this invention, and that the use of the terms “biologically active agent” and “exendin-4 or exenatide based agent” include the use of two or more such agents.

The term “microprojection”, as used herein, refers to one or more piercing elements which are adapted to pierce or cut through the stratum corneum into the underlying epidermis layer, or epidermis and dermis layers, of the skin of a living animal, particularly a mammal and more particularly a human.

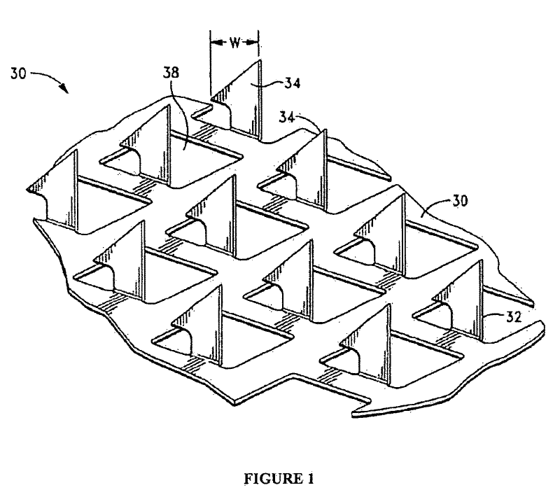

In one embodiment of the invention, the piercing elements have a projection length less than 1000 microns. In a further embodiment, the piercing elements have a projection length of less than 500 microns. In other embodiments, the piercing elements have a projection length of less than 250 microns. The microprojections further have a width (designated “W” in FIG. 1) in the range of about 25 to about 500 microns and a thickness in the range of about 10 to about 100 microns. The microprojections may be formed in different shapes, such as needles, blades, pins, punches, and combinations thereof.

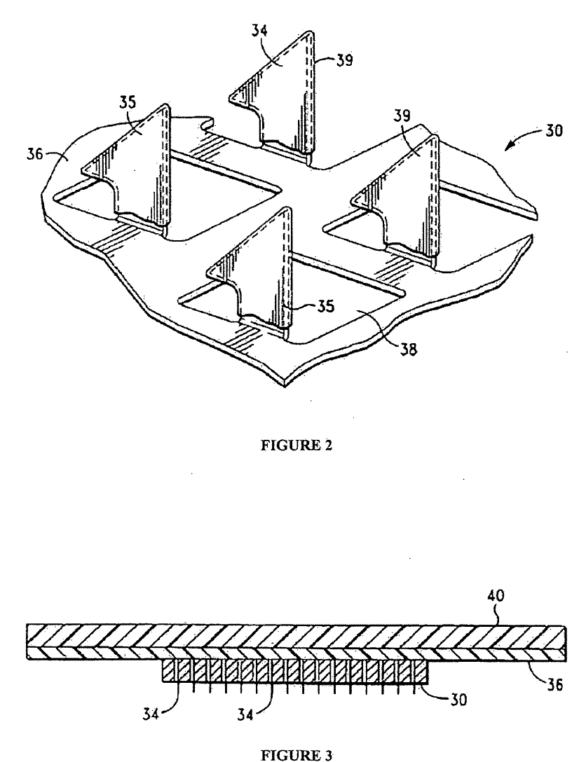

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 2. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “drug coating formulation”, as used herein, is meant to mean and include a freely flowing composition or mixture that is employed to coat the microprojections and/or arrays thereof. Generally, the drug coating formulation includes at least one biologically active agent, which can be in solution or suspension in the formulation.

The term “controlled release coating formulation”, or “sustained release coating formulation” as used herein, includes a freely flowing composition or mixture that is employed to coat the microprojections and/or arrays thereof on top of at least one drug coating. In some embodiments, the controlled release coating formulation includes at least one polymer which imparts the controlled release, for example sustained release properties to the coating.

The terms “biocompatible coating”, “solid coating”, “drug coating” and “dry coating” are interchangeable and, as used herein, are meant to mean and include a “drug coating formulation” in a substantially dry and/or solid state. It is to be understood that all components designated as being present in the “drug coating formulation” are also considered as being disclosed to be in the “biocompatible coating”, “solid coating”, “drug coating” or “dry coating”. The coatings of the present invention include amorphous glassy coatings.

As indicated above, the present invention generally comprises a delivery system including microprojection member (or system) having a plurality of microprojections (or array thereof) that are adapted to pierce through the stratum corneum into the underlying epidermis layer, or epidermis and dermis layers.

In certain embodiments, the present invention provides a transdermal delivery device for delivering a biologically active agent (e.g., an exendin-4 or exenatide based agent) comprising at least one stratum corneum-piercing microprojection, wherein said microprojection has a first coating comprising said biologically active agent and a second coating comprising a polymer, wherein the polymer coating allows controlled release of said biological agent after the transdermal delivery device is applied to the skin of a subject.

In some embodiments, the present invention provides for a transdermal delivery device for delivering a biologically active agent (e.g., an exendin-4 or exenatide based agent) comprising at least one stratum corneum-piercing microprojection, wherein said microprojection has a plurality of coating layers; wherein at least one coating layer comprises said biologically active agent (e.g., an exendin-4 or exenatide based agent) and at least one coating layer comprises a polymer, wherein the polymer coating allows controlled release of said biological agent after the transdermal delivery device is applied to the skin of a subject. In some embodiments, the coating layers comprising the biologically active agent and coating layers comprising the controlled release polymer are alternately disposed on said microprojection.

In one embodiment of the invention, the thickness of the biocompatible coating is less than about 25 microns. In a more specific embodiment, the thickness of the biocompatible coating is less than 10 microns. Thickness of the biocompatible coating is measured from the microprojection surface.

In certain embodiments, the polymer or controlled release polymer used in the polymer coating is a hydrophilic polymer or a hydrophobic PLGA copolymer. In various embodiments of the present invention, the polymer layer has a thickness selected to provide a predetermined sustained release profile for the biologically active agent. In some embodiments, the polymer layer has a copolymer molar mass, a copolymer architecture, a water hydration rate, and/or or layer thickness selected to provide a predetermined sustained release profile for the biologically active agent.

Encapsulation of therapeutic peptides/proteins with PLGA copolymers are traditionally prepared by an emulsification process. As described herein, encapsulation of a peptide/protein for coating on microprojection arrays may require an alternate method as emulsification may be not suitable process for the microprojection array coating methodology. Emulsification process often yields particulates in the μm size range, which may be too large to coat on the microprojections. Furthermore emulsions are inherently unstable systems and to prevent the particulates from coalescence, they need to be suspended in a high viscosity medium and the emulsion may need to be vigorously stirred in a continuous manner.

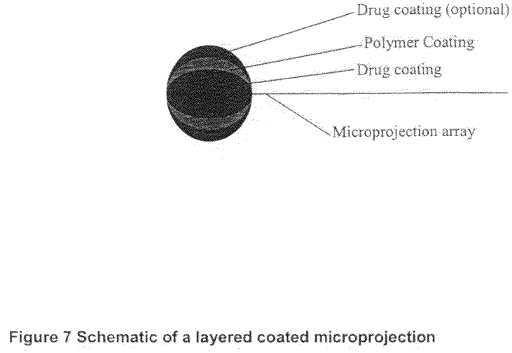

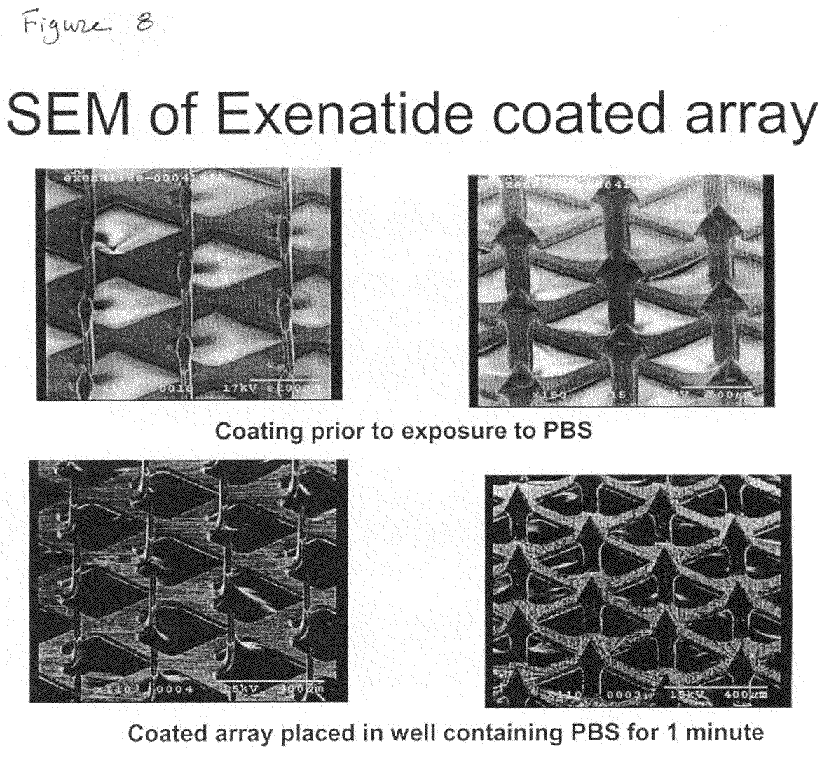

One alternative provided herein is a two step coating process is proposed. A peptide/protein is coated onto a microprojection. Next a secondary coating, for example of a PLGA copolymer is applied utilizing a secondary reservoir. The thickness of the PLGA coating can be controlled by the number of coats and/or concentration of copolymer. The rate of drug release is controlled by the thickness of the copolymer coating, molar mass of copolymer, copolymer chain architecture and type of solvent utilized to dissolve copolymer. Optionally, another coating of the therapeutic drug can be applied to PLGA, thus creating an initial burst dose of the drug. A schematic of the coating is shown in FIG. 7.

In certain embodiments, the device provided herein has a controlled release profile with a shorter tₘₐₓ and a rapid concentration drop off. In some embodiments of the present invention, the controlled release profile of a device has a reduced Cₘₐₓ and an extended drop-off tail. It is to be understood that these pharmacokinetic values are compared to those of standard applications of the biologically active agent.

In certain embodiments, the polymer layer encapsulates the biologically active agent and slows down release of the biologically active agent.

The desired coating thickness of the drug layer is dependent upon several factors, including the required dosage and, hence, coating thickness necessary to deliver the dosage, the density of the microprojections per unit area of the sheet, the viscosity and concentration of the coating composition and the coating method chosen.

In certain embodiments, the present invention provides a device having one or more stratum corneum-piercing microprojections extending therefrom. The microprojections have a dry coating (or biocompatible coating) thereon which contains a biologically active agent. On the dry coating containing the biologically active agent is a controlled release coating. In some embodiments, the controlled release coating is a sustained release coating. Controlled release coatings are applied to dry coating containing the biologically active agent in any manner known in the art. In some embodiments, the controlled release coating is applied in a manner consistent with any of the methods described herein for applying the biocompatible coating.

In some embodiments, the controlled release coating formulation includes at least one polymer that imparts the controlled release (e.g., sustained release) properties to the coating. Polymers that impart controlled release properties to the coating include, by way of non-limiting example, poly(lactic-co-glycolic acid) (PLGA), polycaprolactone, polyglycolide, polylactic acid, poly-3-hydroxybutyrate, polyglycolic acids (PGA) and polylactic acids (PLA), poly(DL-lactic acid-co-glycolic acid) (DL PLGA), poly(D-lactic acid-coglycolic acid) (D PLGA) and poly(L-lactic acid-co-glycolic acid) (L PLGA), poly(ε-caprolactone), poly(ε-caprolactone-co-lactic acid), poly(ε-caprolactone-co-glycolic acid), poly(β-hydroxy butyric acid), poly(alkyl-2-cyanoacrilate), hydrogels such as poly(hydroxyethyl methacrylate), polyamides, poly(amino acids) (e.g., L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, polyvinyl pyrrolidone (e.g., PVP K30), and copolymers thereof. In specific embodiments, the controlled release polymer is PLGA. In more specific embodiments, the PLGA has a molecular weight of 5-15 kDa.

The layered coating approach outlined in FIG. 7 can be applied to aqueous based systems. A peptide/protein is coated onto a microprojection. Next a secondary coating of e.g. Pluronic F127, Povidone (C30), Dextran (67 kDa) polymer is applied, utilizing a secondary reservoir. Generally, low temperatures are employed during the coating process, and due to the viscous nature of the polymers employed and the short residence time of the drug coated microprojections in the aqueous polymeric coating solution dissolution of the drug may be minimal.

In the case of Pluronic F127, this would form a gel once it is hydrated by interstitial fluids, this gel would then control the release rate of the drug due to the increase in diffusion path length. The other polymeric materials do not form gels, but will form a highly viscous layer around the drug core, thus retard the rate of dissolution of the drug.

Other embodiments involve the formation of hydrophilic matrices. A special class of gels, known as thermo-reversible gels (also known by the trademarked name Pluronic), are characterized by the property of being a solid below a critical solution temperature and becoming viscous, or gel-like, above the critical solution temperature. This is in contrast with normal matter that is solid below a critical temperature, for example, the freezing temperature and liquid above that critical temperature, exhibiting decreased viscosity as the temperature of the matter increases. The critical solution temperature of these gels can be tailored by their chemistry such that they are liquid at room temperature or below (0-23° C.) and gel at body temperature (37° C.).

These properties allow formulation of a liquid formulation for coating at ambient temperature. When delivered into the skin at the body temperature, the dry coat hydrolyzes to form a gel, which may slow down the rate of release of the peptide from the gel compared to the case where the dry coat is dissolved outright.

One embodiment contemplates the use of Pluronic F127. Since the Pluronic F127 is a surfactant, the coating solution may be highly wettable and could encounter a high degree of contamination during the coating process. Several aspects are considered in designing coatings based on Pluronic F127. High % of copolymer may be desirable relative to the amount of active. The drug and copolymer are uniformly mixed, achieving controlled release is obtained by considering the solubility of the two components, F127 and drug.

In some embodiments, the biologically active agent is selected from an exendin-4 or exenatide based agent. In certain embodiments, the exenatide based agent is exenatide. In some embodiments, the exenatide based agent is an exenatide salt. In specific embodiments, the exenatide salt comprises a non-volatile counter-ion.

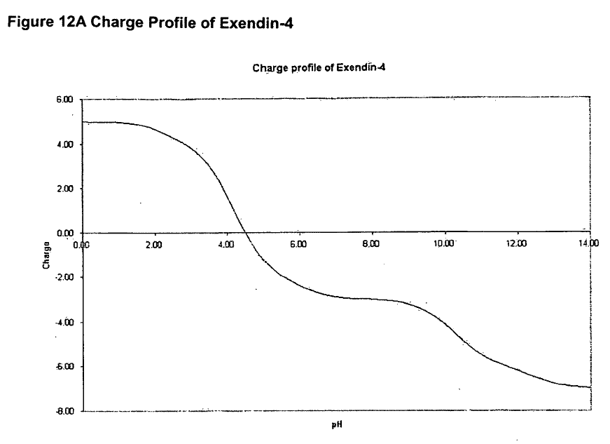

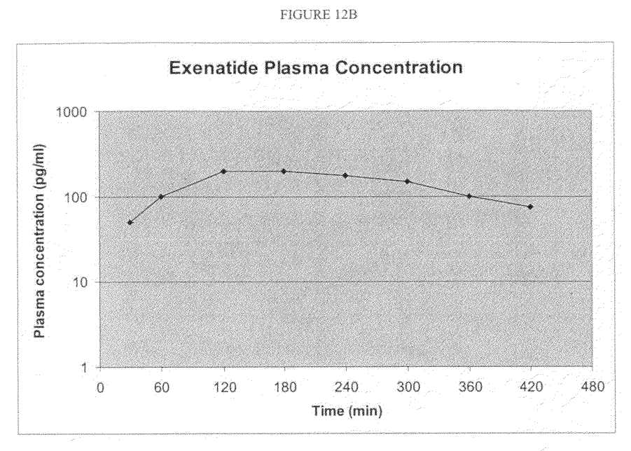

Exendin-4 is a polypeptide with 39 amino acids and a molecular weight of 4187.6 Da. The charge profile of exendin-4 is shown in FIG. 12. Exendin-4 has 5 basic pKas and 7 acidic pKas, and at pH 4.4, the polypeptide presents a zero net electric charge.

In some embodiments, the first coating (or coating comprising the biologically active agent) comprises a first and a second exenatide based agent. In some embodiments, the second exenatide based agent is a second exenatide salt, wherein the second exenatide salt comprises a volatile counter-ion. In specific embodiments, the first exenatide based agent is a first exenatide salt and the second exenatide based agent is a second exenatide salt. In more specific embodiments, the first exenatide salt is a non-volatile salt and the second exenatide salt is a volatile salt.

In other embodiments, the first exenatide based agent is an exenatide salt and the second exenatide based agent is a net neutral species of exenatide. In certain embodiments, the net neutral species of exenatide is obtained from an exenatide salt comprising a volatile counter-ion upon volatilization of the volatile counter-ion. In other embodiments, the net neutral species of exenatide is simply combined with the first exenatide based agent (e.g., an exenatide salt).

In certain embodiments, the present invention provides for a transdermal delivery device comprising at least one stratum corneum-piercing microprojection. In some embodiments, the microprojection has a coating layer; the coating layer has a first exenatide based agent and a second exenatide based agent. In some embodiments, the second exenatide based agent is released in a controlled manner. In certain embodiments, the first exenatide based agent is an exenatide salt with a non-volatile counter-ion and the second exenatide based agent is a net neutral species of exenatide.

Many peptides and polypeptides are prepared as acetate salts. The acetate counterion results in the formation of a species that is soluble, given that the pH of the solution is at least 2 units below or above the pI of the peptide or polypeptide of interest. In the case of the peptide/polypeptide of interest bearing a positive charge addition of a hydrophobic counterion e.g. hexadecanoic acid, pentadecanoic, tetradecanoic, tridecanoic, dodecanoic, decanoic, nonanoic, octanoic, hetpanoic, hexanoic, pentanoic, butanoic acid, and other fatty acids results in the formation of a second species of the biologically active agent that has reduced solubility when the formulation is dried. When a hydrophobic counter ion is employed, a viscosity enhancer is optionally added to the coating formulation.

In the cases of the peptide/polypeptide of interest bearing a negative charge addition of a hydrophobic counterion e.g. ethylamine, propylamine, butylamine would reduce the solubility of the active agent. As a result, the mixed salts of the peptide or polypeptide of interest have different levels of solubility: the peptide/polypeptide molecules associated with the acetate counterion will be soluble and will dissolve rapidly in the interstitial fluid, whilst the second species of the peptide and polypeptide associated with the hydrophobic counterion dissolve at a slower rate to provide sustained blood levels of the agent. The amount of hydrophobic counterion in the coating formulation should represent no more than 99%, preferably no more than 50%, of the amount necessary to neutralize the charge present on the peptide and polypeptide of interest at the pH of the formulation.

The acetate salt of the peptide/polypeptide will contribute to fast onset and the hydrophobic salt of the peptide/polypeptide will stay on for a longer period of time to initiate the phase of extended release. The response of the Cmax and the rate of the concentration drop-off phase can be theoretically varied by the species and relative concentration of the counterions.

Referring now to FIG. 2, there is shown one embodiment of a microprojection member 30 for use with the present invention. As illustrated in FIG. 2, the microprojection member 30 includes a microprojection array 32 having a plurality of microprojections 34. The microprojections 34 preferably extend at substantially a 90° angle from the sheet, which in the noted embodiment includes openings (holes) 38.

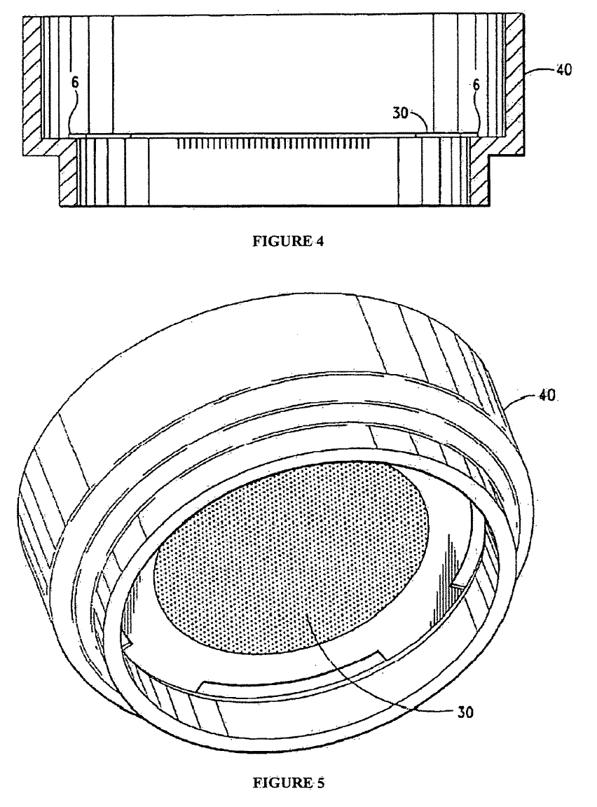

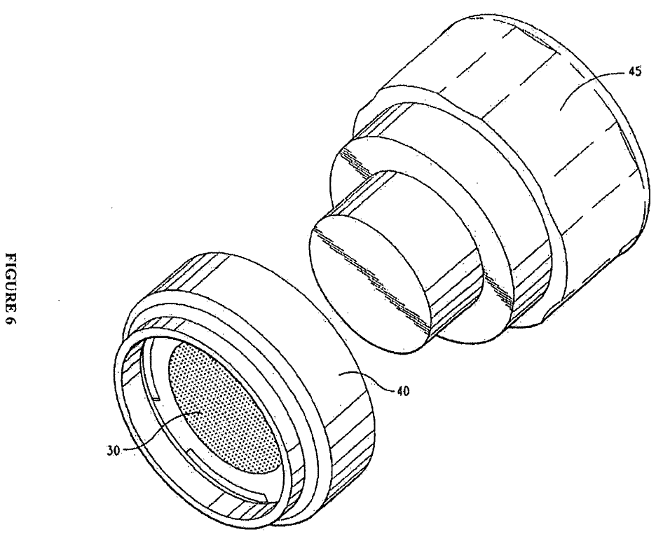

According to the invention, the sheet 36 can be incorporated into a delivery patch, including a backing 40 for the sheet 36, and can additionally include adhesive 16 for adhering the patch to the skin (see FIG. 4). In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microprojection density of at least approximately 10 microprojections/cm². In a more specific embodiment, the microprojection member has in the range of about 200 to about 2000 microprojections/cm². In certain embodiments, the number of openings per unit area through which the agent passes is at least about 10 openings/cm² and less than about 2000 openings/cm².

As indicated, in certain embodiments, the microprojections 34 have a projection length less than 1000 microns. In one embodiment, the microprojections 34 have a projection length of less than 500 microns. In another embodiment, the microprojections have a projection length of less than 250 microns. In some embodiments, the microprojections 34 have a width in the range of about 25 to about 500 microns and thickness in the range of about 10 to about 100 microns.

In further embodiments of the invention, the biocompatibility of the microprojection member 30 is improved to minimize or eliminate bleeding and irritation following application to the skin of a subject. In specific embodiments, the microprojections 34 have a length of less than 145 microns. In more specific embodiments, the microprojections have a length in the range of about 50 to about 145 microns. In even more specific embodiments, the microprojections have a length in the range of about 70 to about 140 microns. Also, in certain embodiments, the microprojection member 30 has an array with a microprojection density greater than 100 microprojections/cm². In specific embodiments, the microprojection density is in the range of about 200 to about 3000 microprojections/cm². Further details regarding microprojection members having improved biocompatibility are found in U.S. Publication No. 20060204562, published Sep. 14, 2006, which is hereby incorporated by reference in its entirety.

In some embodiments, the microprojection member 30 is manufactured from one or more metals, including, by way of non-limiting example, stainless steel, titanium, nickel titanium alloys, or similar biocompatible materials.

In other embodiments of the present invention, the microprojection member 30 is constructed out of a non-conductive material, such as, by way of non-limiting example, a polymeric material. In some embodiments, the microprojection member is coated with a non-conductive material, such as Parylene®, or a hydrophobic material, such as Teflon®, silicon or other low energy material. The noted hydrophobic materials and associated base (e.g., photoresist) layers are set forth in U.S. Application No. 60/484,142, which is incorporated by reference herein in its entirety.

Microprojection members that are employed with various embodiments of the present invention include, by way of non-limiting example, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

In certain embodiments of the invention, the microprojections 34 are configured to reduce variability in the applied coating 35. In some embodiments, suitable microprojections comprise a location having a maximum width transverse to the longitudinal axis that is located at a position in the range of approximately 25% to 75% of the length of the microprojection from the distal tip. Proximal to the location of maximum width, the width of the microprojection tapers to a minimum width. Further details regarding the noted microprojection configurations are found in U.S. Application Ser. No. 60/649,888, filed Jan. 31, 2005, which is incorporated by reference herein in its entirety.

Referring now to FIG. 3, there is shown a microprojection member 30 having microprojections 34 that include a biocompatible coating 35 that includes one or more biologically active agents. In some embodiments, the biologically active agent is selected from an exendin-4 or exenatide based agent. In some embodiments, the biologically active agents are two or more exendin-4 or exenatide based agents.

In certain embodiments of the present invention, the coating 35 partially or completely covers each microprojection 34. In specific embodiments, the coating 35 is in a dry pattern coating on the microprojections 34. In some embodiments, the coating 35 is applied before or after the microprojections 34 are formed.

According to various embodiments of the present invention, the coating 35 is applied to the microprojections 34 by a variety of known methods. In one embodiment, the coating is only applied to those portions the microprojection member 30 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to some embodiments of the invention, the microprojections 34 are adapted to receive and/or enhance the volume of the coating 35. In order to receive and/or enhance the volume of the coating 35, some embodiments of the present invention provide for modification of the microprojections. Modifications include, by way of non-limiting example, apertures (not shown), grooves (not shown), surface irregularities (not shown) or similar modifications. These modifications provide increased surface area upon which a greater amount of coating is deposited.

A further coating method that is employed within the scope of the present invention comprises spray coating. According to the invention, spray coating includes formation of an aerosol suspension of the coating composition. In one embodiment, an aerosol suspension having a droplet size of about 10 to about 200 picoliters is sprayed onto the microprojections 10 and then dried.

In some embodiments, pattern coating is employed to coat the microprojections 34. In certain embodiments, the pattern coating is applied using a dispensing system for positioning the deposited liquid onto the microprojection surface. In some embodiments, the quantity of the deposited liquid is in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

In some embodiments of the present invention, microprojection coating formulations or solutions are applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

In some embodiments, for storage and application, the microprojection member is suspended in a retainer ring by adhesive tabs, as described in detail in U.S. application Ser. No. 09/976,762 (Pub. No. 2002/0091357), which is incorporated by reference herein in its entirety.

In some embodiments, after placement of the microprojection member in the retainer ring, the microprojection member is applied to the patient's skin. In some embodiments, the microprojection member is applied to the patient's skin using an impact applicator as described in Co-Pending U.S. application Ser. No. 09/976,978, which is incorporated by reference herein in its entirety.

As indicated, according to one embodiment of the invention, the drug coating formulations applied to the microprojection member to form solid biocompatible coatings comprises aqueous and non-aqueous formulations having at least one biologically active agent. According to one embodiment of the invention, the biologically active agent is dissolved within a biocompatible carrier or suspended within the carrier.

In specific embodiments, the biologically active agent is an exenatide based agent. In some embodiments, the exenatide based agent is selected from a net neutral species of exenatide, a salt of exenatide, an exenatide analog (or a salt thereof) and a related peptide (or a salt thereof).

In some embodiments of the present invention, the salt of exenatide is a pharmaceutically acceptable salt thereof. Examples of suitable exenatide salts include, by way of non-limiting example, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, \tilde{□}hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

In certain embodiments of the present invention, the salt or salts of exenatide comprise one or more volatile counter-ion.

Volatile counter-ions are defined as weak acids presenting at least one pKa higher than about 2 and a melting point lower than about 50° C. or a boiling point lower than about 170. degree. C. at Pₐₜₘ. Examples of such acids include acetic acid, propionic acid, pentanoic acid and the like. Volatile counter-ions are also defined as weak bases presenting at least one pKa lower than about 12 and a melting point lower than about 50° C. or a boiling point lower than about 170° C. at Pₐₜₘ. Examples of such bases include ammonia and morpholine.

In some embodiments of the present invention, the salt of exenatide comprises one or more non-volatile counter-ion.

Non-volatile counter-ions are defined as weak acids presenting at least one acidic pKa and a melting point higher than about 50° C. or a boiling point higher than about 170° C. at Pₐₜₘ. Examples of such acids include citric acid, succinic acid, glycolic acid, gluconic acid, glucuronic acid, lactic acid, malic acid, pyruvic acid, tartaric acid, tartronic acid, and fumaric acid. Non-volatile counter-ions are also defined as acidic zwitterions presenting at least two acidic pKa, and at least one basic pKa, so that there is at least one extra acidic group as compared to the number of basic groups. Examples of such compounds include glutamic acid and aspartic acid.

Non-volatile counter-ions are also defined as weak bases presenting at least one basic pKa and a melting point higher than about 50° C. or a boiling point higher than about 170° C. at Pₐₜₘ. Examples of such bases include monoethanolomine, diethanolamine, triethanolamine, tromethamine, methylglucamine, glucosamine. Non-volatile counter-ions are also defined as basic zwitterions presenting at least one acidic pKa, and at least two basic pKa's, wherein the number of basic pKa's is greater than the number of acidic pkA's. Examples of such compounds include lysine, arginine, and histidine.

Non-volatile counter-ions are also defined as strong acids presenting at least one pKa lower than about 2. Examples of such acids include hydrochloric acid, hydrobromic acid, nitric acid, sulfonic acid, sulfuric acid, maleic acid, phosphoric acid, benzene sulfonic acid and methane sulfonic acid. Non-volatile counter-ions are further defined as strong bases presenting at least one pKa higher than about 12. Examples of such bases include sodium hydroxide, potassium hydroxide, calcium hydroxide, and magnesium hydroxide.

When referring to the volatility of a counter-ion, reference will always be made to the volatility of the non-ionized form of the counter-ion (e.g., acetic acid versus acetate).

Agents that behave like strong bases or strong acids (e.g., quaternary ammonium salts such as clidinium bromide or glycopyrrolate, sulfate derivatives, such as pentosan polysulfate, some phosphoric derivatives such as nucleic acids) generally are totally ionized in a wide range of pH (i.e. 4-10). The noted pH range covers conditions commonly used with pharmaceutical formulations.

Other compounds, such as neutral polysaccharides (e.g., inulin and dextrans), do not present acidic or basic functions. Since solubility in water is not significantly affected by pH for such classes of agents, they are generally not suitable for practicing the invention.

Conversely, many agents behave as weak acids or weak bases. Their neutral species usually present low water solubility. For example, the neutral species of many peptides, such as exenatide, are insoluble in water. These compounds exhibit maximum solubility in water when they are in an electrically charged state. Because of their weakly acidic or basic nature, the respective concentrations of the neutral and ionized species, and therefore the solubility in water, is pH dependant. The invention applies to this class of agents.

Accordingly, the invention includes compositions of a biologically active agent with a non-volatile counter-ion sufficient to minimize the presence of the neutral form of the agent to assure enhanced solubility of the agent in the formulation, stability during storage in the solid state, and dissolution in the biological fluids at the time of administration.

In some embodiments of the present invention, the amount of counter-ion present is an amount necessary to neutralize the charge present on the agent at the pH of the formulation. In certain embodiments, excess of counter-ion (as the free acid or base or as a salt) can be added to the agent in order to control pH and to provide adequate buffering capacity.

In some embodiments of the present invention, the salts of exenatide present in the coating formulation comprise one or more volatile counter-ion and one or more non-volatile counter-ion.

In some embodiments of the present invention, the amount of non-volatile counter-ion in the coating formulation is less than about 99% of the total amount of counter-ion present in the coating formulation. In more specific embodiments, the amount of non-volatile counter-ion in the coating formulation is less than about 90% of the total amount of counter-ion present in the coating formulation. In some embodiments of the present invention, the amount of non-volatile counter-ion in the coating formulation is less than about 99% of the amount of counter-ion necessary to neutralize the charge present on the agent at the pH of the coating formulation. In more specific embodiments, the amount of non-volatile counter-ion in the coating formulation is less than about 90% of the amount of counter-ion necessary to neutralize the charge present on the agent at the pH of the coating formulation. In some embodiments, the amount of volatile counter-ion in the coating formulation is more than about 1% of the total amount of counter-ion present in the coating formulation. In more specific embodiments, the amount of volatile counter-ion in the coating formulation is more than about 10% of the total amount of counter-ion present in the coating formulation. In some embodiments of the present invention, the amount of volatile counter-ion in the coating formulation is more than about 1% of the amount of counter-ion necessary to neutralize the charge present on the agent at the pH of the coating formulation. In more specific embodiments, the amount of volatile counter-ion in the coating formulation is more than about 10% of the amount of counter-ion necessary to neutralize the charge present on the agent at the pH of the coating formulation.

Following coating and drying, a substantial fraction of the volatile counter-ion is lost. This, in turn, results in formation of less charged and less water soluble species in the solid formulation.

In certain embodiments of the present invention, the coating formulation comprises a volatile solvent. Volatile solvents include, by way of non-limiting example, water, ethanol, isopropanol, methanol, benzene, acetone, ethyl ether, and the like, and mixtures thereof.

In alternative embodiments, a similar result is achieved by combining an exenatide salt comprising a non-volatile counter-ion with a net neutral species of exenatide. In some embodiments, the amount of the exenatide salt comprising a non-volatile counter-ion represents less than about 99% of the total molar amount of exenatide. In more specific embodiments, the amount of exenatide salt comprising a non-volatile counter-ion represents less than about 90% of the total molar amount of exenatide. In some embodiments, the amount of net neutral species represents more than about 1% of the total molar amount of exenatide. In more specific embodiments, the amount of net neutral species represents more than about 10%, of the molar fraction of the exenatide. In some embodiments, the mixture is solubilized or suspended in an adequate coating volatile solvent. Suitable volatile solvents include, by way of non-limiting example, water, ethanol, isopropanol, methanol, benzene, acetone, ethyl ether, and the like, and mixtures thereof.

In both cases, the charged species of the biologically active agent quickly dissolves when the microprojection member is applied to the patient, providing a bolus delivery that results in rapid elevation of the agent to therapeutically relevant blood levels. In turn, the reduced solubility species allows sustained delivery of the biologically active agent, providing delivery that maintains a therapeutically relevant blood level for a desired period of time.

In certain embodiments of the present invention, an exenatide based agent is formulated for transdermal delivery to provide treatment of diabetes or obesity. In specific embodiments, the diabetes is type 2 diabetes or NIDDM.

In some embodiments of the present invention, the delivery system and drug coating formulation described herein provide for a pharmacokinetic profile in humans that includes the establishment of therapeutically relevant blood levels of a biologically active agent (e.g., exenatide or an exenatide based agent) in less than 2 hours. In more specific embodiments, the pharmacokinetic profile in humans includes the establishment of a therapeutically relevant blood level in less than 30 minutes. In some embodiments, the therapeutically relevant blood levels are sustained for about 2 hours, about 6 hours, about 12 hours and about 24 hours. In other embodiments, the therapeutically relevant blood levels are sustained for about a week. In some embodiments, the therapeutically relevant blood levels are sustained for between about 2 hours and about a month. In some embodiments of the present invention, the total dose of a biologically active agent (e.g., exenatide or an exenatide based agent) delivered transdermally is in the range of about 1 μg to about 100 μg per day. In specific embodiments, the total dose of the biologically active agent (e.g., exenatide or an exenatide based agent) delivered is in the range of about 2 to about 30 μg per day. In more specific embodiments, the total dose of the biologically active agent (e.g., exenatide or an exenatide based agent) delivered is in the range of about 5 to about 20 μg per day. In some embodiments, the total dose of the biologically active agent (e.g., exenatide or an exenatide based agent) delivered is in the range of about 2.5 to about 10 μg per day. In certain embodiments, the total dose of biologically active agent (e.g., an exenatide based agent) administered by a device of the present invention is between about 1 μg and about 5 mg. In specific embodiments, the total dose administered by a device of the present invention is about 2 to about 30 μg. In other embodiments, the total dose administered by a device of the present invention is about 0.5 mg to about 3 mg. In some embodiments, the total dose administered by a device of the present invention is about 2 mg.

When administered by standard subcutaneous means, exenatide is rapidly absorbed, having a tₘₐₓ of about 2 hours. Standard subcutaneous administration involves injection of a sterile solution containing exenatide in a concentration of 250 μg/mL. Administration doses are typically in 5 μg and 10 μg doses. Subcutaneous administration of a 10 μg dose of exenatide achieves a Cₘₐₓ of about 200 to about 250 pg/mL and has an AUC of about 1000 to about 1200 pg·h/mL. The mean clearance of exenatide in humans is 9.1 L/h and the mean terminal half-life of exenatide is about 2.4 hours, independent of dose.

In certain embodiments, the formulations and/or devices described herein provide for shorter tₘₐₓ values of a biologically active agent (e.g., exenatide or an exenatide based agent) than those of standard subcutaneous administration of the biologically active agent. In some of these embodiments, the shortened tₘₐₓ is followed by a rapid concentration drop off. In some embodiments of the present invention, the formulations and/or devices described herein provide for reduced Cₘₐₓ values of a biologically active agent (e.g., exenatide or an exenatide based agent) than those of standard subcutaneous administration. In some of these embodiments, the reduced Cₘₐₓ is accompanied by and an extended drop-off tail

In one embodiment, the invention includes a formulation of volatile and non-volatile counter-ions with a exenatide-based agent. For example, the exenatide-based agent is mixed with an equimolar amount of the volatile counter-ion (e.g., acetic acid) and the non-volatile counter-ion (e.g., tartaric acid). Upon coating, some of the acetic acid will volatilize leaving a solid coating of exenatide base on the microprojections and substantially no tartaric acid will volatilize leaving a solid coating of exenatide tartarate on the microprojections. Upon administration into a patient, the exenatide tartarate will exhibit improved solubility and promote the fast onset of action. Correspondingly, the exenatide base will exhibit reduced solubility to yield a prolonged therapeutic effect.

In some embodiments, the solid coating is obtained by drying a formulation on the microprojection as described in U.S. Patent Application Publication No. 2002/0128599, which is hereby incorporated by reference in its entirety. In other embodiments, other suitable processes are employed. In some embodiments, during the drying process, all volatiles, including water are removed. In other embodiments, the final solid or “dry” coating still contains up to about 10% water after drying.

As is known in the art, the kinetics of the agent-containing coating dissolution and release will depend on many factors including the nature of the agent (including the nature of the counter-ion), the coating process, the coating thickness and the coating composition (e.g., the presence of coating formulation additives). In some embodiments, the nature of the release kinetics profile, makes it necessary to maintain the coated microprojections in piercing relation with the skin for extended periods of time (e.g., up to about 8 hours). This can be accomplished by anchoring the delivery device to the skin using adhesives or by using anchored microprojections such as described in WO 97/48440, incorporated by reference in its entirety.

In certain specific embodiments, the present invention provides a device having a plurality of stratum corneum-piercing microprojections extending therefrom. The microprojections are adapted to pierce through the stratum corneum into the underlying epidermis layer, or epidermis and dermis layers, but do not penetrate so deep as to reach the capillary beds and cause significant bleeding. The microprojections have a dry coating (or biocompatible coating) thereon which contains a biologically active agent (e.g., an exenatide based agent). The coating is formulated to contain a non-volatile counter-ion to create an ionic species of exenatide that has enhanced solubility upon piercing the skin. Additionally, the coating contains a volatile counter-ion to create a species of the exenatide that has reduced solubility.

In some embodiments, the dry (or biocompatible) coating contains a pharmaceutically acceptable salt of exenatide. In certain embodiments, the dry coating is substantially free of net neutral species of exenatide.

In certain embodiments, the pharmaceutically acceptable salt of exenatide is prepared by adding exenatide and an acid or base to the coating formulation. In other embodiments, the pharmaceutically acceptable salt of exenatide is prepared separately from the coating formulation and added as the salt to the coating formulation.

In some embodiments, the biologically active agent (e.g., an exenatide based agent) is present in the drug coating formulation at a concentration in the range of about 1 to about 30 wt. %. In more specific embodiments, the biologically active agent (e.g., an exenatide based agent) is present in the drug coating formulation at a concentration in the range of about 10 to about 20 wt. %.

In certain embodiments, the amount of biologically active agent (e.g., an exenatide based agent) contained in the biocompatible coating on the plurality of microprojection member is in the range of about 1 μg to about 5 mg. In specific embodiments, the amount of a biologically active agent (e.g., an exenatide based agent) contained in the dry coating on the microprojection is in the range of about 2 to about 100 μg. In more specific embodiments, the amount of (e.g., an exenatide based agent) contained in the dry coating on the microprojection is in the range of about 5 to about 20 μg. In other embodiments, the amount of a biologically active agent (e.g., an exenatide based agent) is in the range of about 1 mg to about 3 mg. In specific embodiments, the amount of biologically active agent (e.g., an exenatide based agent) is present in about 2 mg.

In some embodiments of the present invention, the drug coating includes at least one buffer. Examples of such buffers include, by way of non-limiting example, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, γ-hydroxybutyric acid, crotonic acid, angelic acid, hydracrylic acid, aspartic acid, glutamic acid, glycine and mixtures thereof.

In one embodiment of the invention, the drug coating formulation includes at least one antioxidant. In certain embodiments, the antioxidant is a sequestering agent. Sequestering agents include, by way of non-limiting example, sodium citrate, citric acid, EDTA (ethylene-dinitrilo-tetraacetic acid). In other embodiments, the antioxidant is a free radical scavenger. Free radical scavengers include, by way of non-limiting example, ascorbic acid, methionine, sodium ascorbate and the like. In specific embodiments, the antioxidant is selected from EDTA and/or methionine. In certain embodiments of the present invention, the antioxidant is present in the drug coating formulation in the range of about 0.01 to about 20 wt %. In specific embodiments, the antioxidant is present in the drug coating formulation in an amount from about 0.03 to about 10 wt. %.

In one embodiment of the invention, the drug coating formulation includes at least one surfactant, which can be zwitterionic, amphoteric, cationic, anionic, or nonionic. Surfactants include, by way of non-limiting example, sodium lauroamphoacetate, sodium dodecyl sulfate (SDS), cetylpyridinium chloride (CPC), dodecyltrimethyl ammonium chloride (TMAC), benzalkonium, chloride, polysorbates, such as polysorbate (Tween) 20 and polysorbate (Tween) 80, other sorbitan derivatives, such as sorbitan laurate, alkoxylated alcohols, such as laureth-4 and polyoxyethylene castor oil derivatives, such as Cremophor EL®. In one embodiment of the invention, the concentration of the surfactant is in the range of about 0.01 to about 20 wt. % of the drug coating formulation. In a specific embodiment, the surfactant is in the range of about 0.05 to about 1 wt. % of the drug coating formulation.

In some embodiments of the invention, the drug coating formulation includes at least one polymeric material or polymer that has amphiphilic properties, which can comprise, without limitation, cellulose derivatives, such as hydroxyethylcellulose (HEC), hydroxypropylmethylcellulose (HPMC), hydroxypropycellulose (HPC), methylcellulose (MC), hydroxyethylmethylcellulose (HEMC), or ethylhydroxy-ethylcellulose (EHEC), as well as pluronics. In one embodiment of the invention, the concentration of the polymer presenting amphiphilic properties in the drug coating formulation is in the range of about 0.01 to about 20 wt. % of the drug coating formulation. In specific embodiments, the concentration of the polymer presenting amphiphilic properties in the drug coating formulation is in the range of about 0.03 to about 10 wt. % of the drug coating formulation.

In another embodiment, the drug coating formulation includes a hydrophilic polymer selected from, by way of non-limiting example, hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers. In some embodiments, the concentration of the hydrophilic polymer in the drug coating formulation is in the range of about 1 to about 30 wt. % of the drug coating formulation. In more specific embodiments, the concentration of the hydrophilic polymer in the drug coating formulation is in the range of about 1 to about 20 wt. % of the drug coating formulation.

In another embodiment of the invention, the drug coating formulation includes a biocompatible carrier, which can comprise, by way of non-limiting example, human albumin, bioengineered human albumin, polyglutamic acid, polyaspartic acid, polyhistidine, pentosan polysulfate, polyamino acids, sucrose, trehalose, melezitose, raffinose, stachyose, mannitol, and other sugar alcohols. In some embodiments, the concentration of the biocompatible carrier in the drug coating formulation is in the range of about 2 to about 70 wt. %. In specific embodiments, the concentration of the biocompatible carrier in the drug coating formulation is in the range of about 5 to about 50 wt. % of the drug coating formulation.

In another embodiment, the drug coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and inulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

In some embodiments, the concentration of the stabilizing agent in the drug coating formulation is at ratio of about 0.1:1 to about 2:1 with respect to the biologically active agent (e.g., an exenatide-based agent). In specific embodiments, the concentration of the stabilizing agent in the drug coating formulation is at ratio of about 0.25:to about 1.0:1 with respect to the biologically active agent (e.g., an exenatide-based agent).

In another embodiment, the drug coating formulation includes a vasoconstrictor. Vasoconstrictors are selected from, by way of non-limiting example, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylbexedrine, pseudoephedrine, tetrahydrozoline, tramazoline, tuaminoheptane, tymazoline, vasopressin, xylometazoline and the mixtures thereof. In specific embodiments, the vasoconstrictors include one or more of epinephrine, naphazoline, tetrahydrozoline indanazoline, metizoline, tramazoline, tymazoline, oxymetazoline and xylometazoline.

As will be appreciated by one having ordinary skill in the art, the addition of a vasoconstrictor to the drug coating formulations and, hence, solid biocompatible coatings of the invention is useful in some embodiments to prevent bleeding that can occur following application of the microprojection member or array. In some embodiments, the vasoconstrictor is also useful to prolong the pharmacokinetics of the biologically active agent (e.g., an exenatide-based agent) through reduction of the blood flow at the application site and reduction of the absorption rate from the skin site into the system circulation. In certain embodiments, the concentration of the vasoconstrictor is in the range of about 0.1 wt. % to about 10 wt. % of the drug coating formulation.

In another embodiment of the present invention, the drug coating formulation includes at least one “pathway patency modulator”. Pathway patency modulators include, by way of non-limiting example, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinaate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the drug coating formulation includes a solubilising/complexing agent. Such agents include, by way of non-limiting example, Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. In specific embodiments, the solubilising/complexing agents are beta-Cyclodextrin, hydroxypropyl beta-Cyclodextrin, 2-hydroxypropyl-beta-Cyclodextrin and/or sulfobutylether7 beta-Cyclodextrin. In certain embodiments, the concentration of the solubilising/complexing agent is in the range of about 1 wt. % to about 20 wt. % of the drug coating formulation.

In another embodiment of the invention, the drug coating formulation includes at least one non-aqueous solvent. Suitable non-aqueous solvents include, by way of non-limiting example, ethanol, isopropanol, methanol, propanol, butanol, propylene glycol, dimethysulfoxide, glycerin, N,N-dimethylformamide, ethyl acetate, and polyethylene glycol 400. In certain embodiments, the non-aqueous solvent is present in the drug coating formulation in the range of about 1 wt. % to about 50 wt. % of the drug coating formulation.

In other embodiments, known formulation adjuvants are also added to the drug coating formulations provided they do not adversely affect the necessary solubility and viscosity characteristics of the drug coating formulation and the physical integrity of the dried coating.

In certain embodiments, the drug coating formulations have a viscosity between about 3 and about 500 centipoise.

In one embodiment of the invention, the thickness of the biocompatible coating is less than about 25 microns. In a more specific embodiment, the thickness of the biocompatible coating is less than 10 microns. Thickness of the biocompatible coating is measured from the microprojection surface.

The desired coating thickness of the drug layer is dependent upon several factors, including the required dosage and, hence, coating thickness necessary to deliver the dosage, the density of the microprojections per unit area of the sheet, the viscosity and concentration of the coating composition and the coating method chosen.

In certain embodiments, the present invention provides a device having one or more stratum corneum-piercing microprojections extending therefrom. The microprojections have a dry coating (or biocompatible coating) thereon which contains a biologically active agent. On the dry coating containing the biologically active agent is a controlled release coating. In some embodiments, the controlled release coating is a sustained release coating. Controlled release coatings are applied to dry coating containing the biologically active agent in any manner known in the art. In some embodiments, the controlled release coating is applied in a manner consistent with any of the methods described herein for applying the biocompatible coating.

In some embodiments, the controlled release coating formulation includes at least one polymer that imparts the controlled release (e.g., sustained release) properties to the coating. Polymers that impart controlled release properties to the coating include, by way of non-limiting example, poly(lactic-co-glycolic acid) (PLGA), polycaprolactone, polyglycolide, polylactic acid, poly-3-hydroxybutyrate, polyglycolic acids (PGA) and polylactic acids (PLA), poly(DL-lactic acid-co-glycolic acid) (DL PLGA), poly(D-lactic acid-coglycolic acid) (D PLGA) and poly(L-lactic acid-co-glycolic acid) (L PLGA), poly(ε-caprolactone), poly(ε-caprolactone-co-lactic acid), poly(ε-caprolactone-co-glycolic acid), poly(β-hydroxy butyric acid), poly(alkyl-2-cyanoacrilate), hydrogels such as poly(hydroxyethyl methacrylate), polyamides, poly(amino acids) (e.g., L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, polyvinyl pyrrolidone (e.g., PVP K30), and copolymers thereof. In specific embodiments, the controlled release polymer is PLGA. In more specific embodiments, the PLGA has a molecular weight of 5-15 kDa.

In accordance with one embodiment of the invention, the method for delivering a biologically compatible agent (e.g., an exenatide based agent) contained in the biocompatible coating on the microprojection member includes the following steps: the coated microprojection member is initially applied to the patient's skin via an actuator, wherein the microprojections pierce the stratum corneum. In some embodiments, the coated microprojection member is left on the skin for a period lasting from 5 seconds to 24 hours. Following the desired wearing time, the microprojection member is removed.

In some embodiments, the amount of biologically compatible agent (e.g., an exenatide based agent) contained in the biocompatible coating (i.e., dose) is in the range of about 1 μg to about 5 mg per dosage unit. In specific embodiments, the amount of biologically compatible agent (e.g., an exenatide based agent) contained in the biocompatible coating is in the range of about 2 to about 100 μg per dosage unit. In more specific embodiments, the amount of biologically compatible agent (e.g., an exenatide based agent) contained in the biocompatible coating is in the range of about 5 to about 30 μg per dosage unit.

In all cases, after a coating has been applied, the coating formulation is dried onto the microprojections 34 by various means. In one embodiment of the invention, the coated microprojection member 30 is dried in ambient room conditions. However, various temperatures and humidity levels can be used to dry the coating formulation onto the microprojections. Additionally, the coated member can be heated, lyophilized, freeze dried or similar techniques used to remove the water from the coating.

It will be appreciated by one having ordinary skill in the art that in order to facilitate drug transport across the skin barrier, certain embodiments of the present invention provide for the administration of the delivery systems provided herein in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169383, the disclosures of which are incorporated by reference herein in their entirety.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported.

Additionally, other transport enhancing methods, such as sonophoresis or piezoelectric devices, can be used in conjunction with the invention.

### EXAMPLES

The following examples are given to enable those skilled in the art to more clearly understand and practice the present invention. They should not be considered as limiting the scope of the invention, but merely as being illustrated as representative thereof.

### Example 1

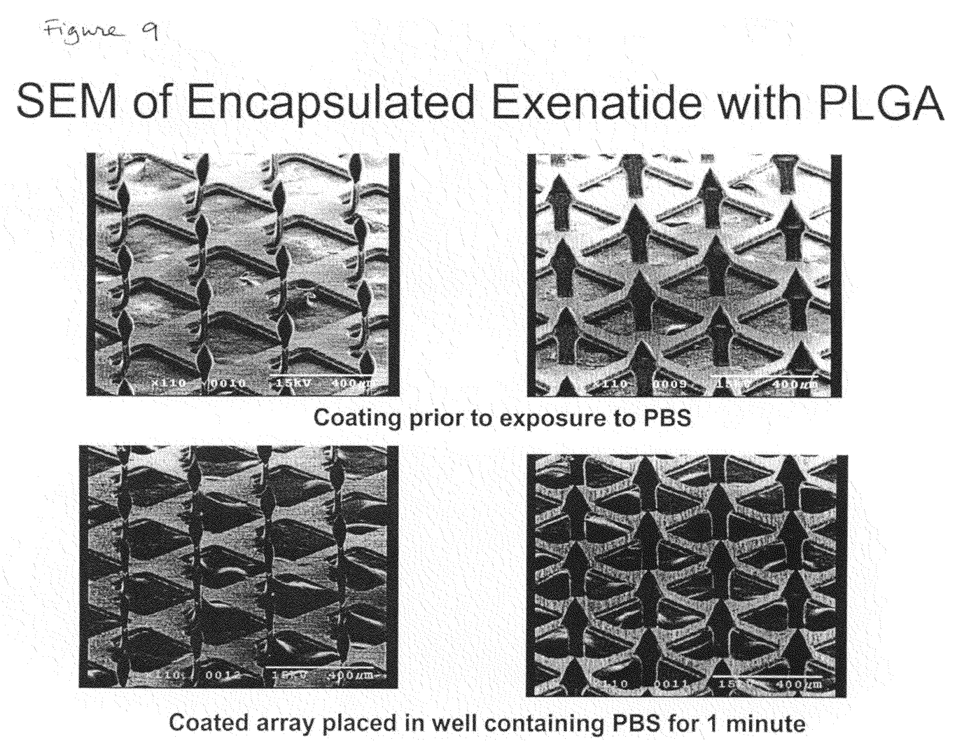

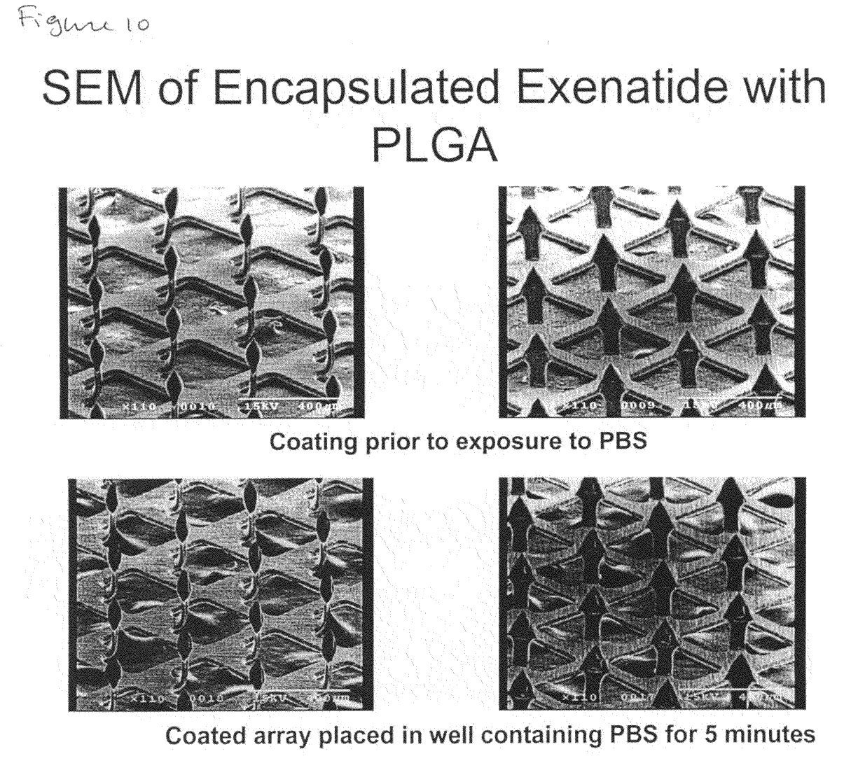

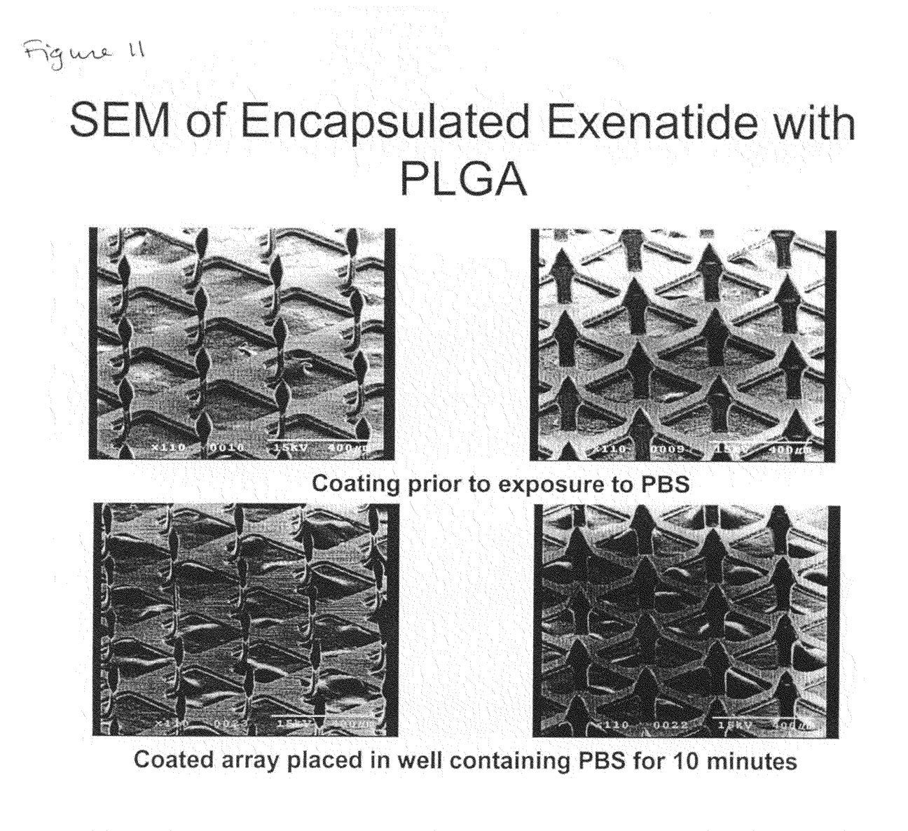

Exenatide coated microprojection arrays were produced with a polymer layer on top of a drug layer containing exenatide. Two reservoirs one containing the exenatide drug formulation and one containing a polymer formulation were used in sequence. The exenatide drug formulation contained 16% w/w exenatide, 16% w/w sucrose, 0.2% w/w HCl and 0.2% w/w polysorbate 20. The polymer formulation contained 500 mg/mL of PLGA in ethyl acetate. A microprojection array with the exenatide drug formulation coating only is shown in FIG. 6. FIG. 7 shows a microprojection array coated with the exenatide formulation with a PLGA polymer coating on top of the exenatide formulation coating. FIG. 8 shows the microprojection array coated with the exenatide drug formulation only after being exposed to PBS for one minute. FIG. 9 shows the microprojection array coated with exenatide drug formulation and a PLGA polymer coating on top of the exenatide formulation coating after being exposed to PBS for 1 minute. FIG. 11 shows the microprojection array coated with exenatide drug formulation and a PLGA polymer coating on top of the exenatide formulation coating after being exposed to PBS for 10 minutes. As can be seen, the microprojection array coated with an unencapsulated exenatide drug formulation shows rapid dissolution in PBS. Encapsulated exenatide drug coatings show much slower dissolution rates.

1. A transdermal delivery device for delivering an exenatide based agent comprising at least one stratum corneum-piercing microprojection, wherein said microprojection has a first coating comprising said exenatide based agent and a second coating comprising a polymer, wherein the polymer coating allows controlled release of said biological agent after the transdermal delivery device is applied to the skin of a subject.

2. A transdermal delivery device for delivering an exenatide based agent comprising at least one stratum corneum-piercing microprojection, wherein said microprojection has a plurality of coating layers; wherein at least one coating layer comprises said exenatide based agent and at least one coating layer comprises a polymer, wherein the polymer coating allows controlled release of said biological agent after the transdermal delivery device is applied to the skin of a subject.

3. The transdermal delivery device of claim 2 wherein coating layers comprising the exenatide based agent and coating layers comprising the controlled release polymer are alternately disposed on said microprojection.

4. The device of claim 1, wherein the polymer is a hydrophilic polymer or a hydrophobic PLGA copolymer.

5-10. (canceled)

11. The device of claim 1, wherein the exenatide based agent is an exenatide salt.

12. The device of claim 11, wherein the exenatide salt comprises a non-volatile counter-ion.

13. The device of claim 12, wherein the first coating further comprises a second exenatide based agent.

14. The device of claim 13, wherein the second exenatide based agent is a second exenatide salt, wherein the second exenatide salt comprises a volatile counter-ion.

15. The device of claim 13, wherein the second exenatide based agent is a net neutral species of exenatide.

16. The device of claim 15, wherein the net neutral species of exenatide is obtained from an exenatide salt comprising a volatile counter-ion and upon volatilization of said volatile counter-ion.

17. A transdermal delivery device comprising at least one stratum corneum-piercing microprojections, wherein said microprojections has a coating layer; wherein the coating layer comprises a first exenatide based agent and a second exenatide based agent, wherein the second exenatide based agent is released in a controlled manner.

18. The device of claim 17, wherein the first exenatide based agent is an exenatide salt with a non-volatile counter-ion and the second exenatide based agent is a net neutral species of exenatide.

19-20. (canceled)

21. A composition for coating a transdermal delivery device having stratum corneum-piercing microprojections comprising a formulation of exenatide based agent, a non-volatile counter-ion and a volatile counter-ion, wherein said non-volatile counter-ion causes the formulation of a first species of exenatide based agent that has improved solubility when the formulation is dried and wherein the volatile counter-ion causes the formation of a second species of exenatide based agent that has reduced solubility when the formulation is dried.

22. The composition of claim 21, wherein said first species is adapted to rapidly provide a therapeutically relevant blood level of said biologically active agent when said formulation is allowed to dissolve in a bodily fluid.

23. The composition of claim 21, wherein said second species is adapted to provide a sustained therapeutically relevant blood level of said biologically active agent when said formulation is allowed to dissolve in a bodily fluid.

24. The composition of claim 21, comprising approximately equimolar amounts of said non-volatile counter-ion and said volatile counter-ion.

25. The composition of claim 21, wherein said formulation has a pH, the exenatide based agent has a positive charge at said formulation pH and said non-volatile counter-ion comprises a non-volatile weak acid.

26. The composition of claim 26, wherein said non-volatile weak acid has an acidic pKa and a property selected from the group consisting of a melting point higher than about 50° C. and a boiling point higher than about 170° C. at atmospheric pressure.

27. The composition of claim 26, wherein said non-volatile weak acid is selected from the group consisting of citric acid, succinic acid, glycolic acid, gluconic acid, glucuronic acid, lactic acid, malic acid, pyruvic acid, tartaric acid, tartronic acid, and fumaric acid.

28. The composition of claim 21, wherein said formulation has a pH, the exenatide based agent has a positive charge at said formulation pH and said non-volatile counter-ion comprises a strong acid.

29. The composition of claim 28, wherein said strong acid has at least one pKa lower than about 2.

30. The composition of claim 29, wherein said strong acid is selected from the group consisting of hydrochloric acid, hydrobromic acid, nitric acid, sulfonic acid, sulfuric acid, maleic acid, phosphoric acid, benzene sulfonic acid and methane sulfonic acid.

31. The composition of claim 21, wherein said formulation has a pH, the exenatide based agent has a positive charge at said formulation pH and said non-volatile counter-ion comprises an acidic zwitterion.

32. The composition of claim 31, wherein said acidic zwitterion has at least two acidic pKas and at least one basic pKa, so that there is at least one acidic pKa more than said basic pKas.

33. The composition of claim 32, wherein said acidic zwitterion is selected from the group consisting of glutamic acid and aspartic acid.

34. The composition of claim 21, wherein said formulation has a pH, the exenatide based agent has a negative charge at said formulation pH and said non-volatile counter-ion comprises a non-volatile weak base.

35. The composition of claim 34, wherein said non-volatile weak base has a basic pKa and a property selected from the group consisting of a melting point higher than about 50° C. and a boiling point higher than about 170° C. at atmospheric pressure.

36. The composition of claim 35, wherein said non-volatile weak base is selected from the group consisting of monoethanolomine, diethanolamine, triethanolamine, tromethamine, methylglucamine, glucosamine.

37. The composition of claim 21, wherein said formulation has a pH, the exenatide based agent has a negative charge at said formulation pH and said non-volatile counter-ion comprises a strong base.

38. The composition of claim 37, wherein said strong base has at least one pKa higher than about 12.

39. The composition of claim 38, wherein said strong base is selected from the group consisting of sodium hydroxide, potassium hydroxide, calcium hydroxide, and magnesium hydroxide.

40. The composition of claim 21, wherein said formulation has a pH, the exenatide based agent has a negative charge at said formulation pH and said non-volatile counter-ion comprises a basic zwitterion.

41. The composition of claim 40, wherein said basic zwitterion has at least two basic pKas and at least one acidic pKa, so that there is at least one basic pKa more than acidic pKas.

42. The composition of claim 41, wherein said basic zwitterion is selected from the group consisting of histidine, lysine, and arginine.

43. The composition of claim 21, wherein said formulation has a pH, the exenatide based agent has a positive charge at said formulation pH and said non-volatile counter-ion comprises a mixture of counter-ions comprising at least one non-volatile strong acid and at least one non-volatile weak acid.

44. The composition of claim 21, wherein said formulation has a pH, the exenatide based agent has a negative charge at said formulation pH and said non-volatile counter-ion comprises a mixture of counter-ions comprising at least one non-volatile strong base and at least one non-volatile weak base.

45. The composition of claim 21, wherein said formulation has a pH, said biologically active agent has a positive charge at said formulation pH and said volatile counter-ion comprises a volatile weak acid.

46. The composition of claim 45, wherein said volatile weak acid has an acidic pKa higher than approximately 2 and a property selected from the group consisting of a melting point lower than about 50° C. and a boiling point lower than about 170° C. at Pₐₜₘ.

47. The composition of claim 46, wherein said volatile weak acid is selected from the group consisting of acetic acid, propionic acid and pentanoic acid.

48. The composition of claim 21, wherein said formulation has a pH, the exenatide based agent has a negative charge at said formulation pH and said volatile counter-ion comprises a volatile weak base.

49. The composition of claim 48, wherein said volatile weak acid has a basic pKa lower than approximately 12 and a property selected from the group consisting of a melting point lower than about 50° C. and a boiling point lower than about 170° C. at Pₐₜₘ.

50. The composition of claim 49, wherein said volatile weak base is selected from the group consisting of ammonia and morpholine.

51-71. (canceled)

72. A method for transdermally delivering an exenatide based agent comprising the steps of: providing a transdermal delivery device having at least one stratum corneum-piercing microprojection, the microprojection including a biocompatible coating comprising a dried formulation of said exenatide based agent, a non-volatile counter-ion and a volatile counter-ion, wherein said non-volatile counter-ion causes the formation of a first species of exenatide based agent that has improved solubility when said formulation is dried and said volatile counter-ion causes the formation of a second species of exenatide based agent that has reduced solubility when said formulation is dried; and applying said delivery device to a patient to deliver said biologically active agent.

73-100. (canceled)
